(19) United States
(12) Patent Application Publication
(10) Pub. No.: US 2025/0205093 A1
(43) Pub. Date: Jun. 26, 2025

(54) STRETCHABLE SHEET STRUCTURE AND DISPOSABLE ABSORBENT PRODUCT

(71) Applicant: LIVEDO CORPORATION, Ehime (JP)

(72) Inventors: Keiichi ISHIKAWA, Tokushima (JP); Osamu MORIURA, Tokushima (JP)

(73) Assignee: LIVEDO CORPORATION, Ehime (JP)

(21) Appl. No.: 18/848,687

(22) PCT Filed: Jul. 27, 2022
(86) PCT No.: PCT/JP2022/028974

§ 371 Date: Sep. 19, 2024

(30) Foreign Application Priority Data
Mar. 25, 2022 (JP) 2022-049269
Apr. 25, 2022 (JP) 2022-071690

(51) Int. Cl. A61F 13/49

(52) U.S. Cl. CPC A61F 13/49019; A61F 13/49017; A61F 2013/49022; A61F 2013/49092

(57) ABSTRACT

A gather sheet (32) having a stretchable sheet structure includes an inside sheet (331), an outside sheet (332), a first elastic member (3221), a second elastic member (3222), and a third elastic members (3223), the first to third elastic members being positioned between the inside sheet (331) and the outside sheet (332) and extending in a stretching direction. The inside sheet (331) and the outside sheet (332) are joined together by a plurality of linear joining units (5) aligned in the stretching direction. Joining widths of each linear joining units (5) at intersecting positions of the linear joining units and the first to third elastic members (3221 to 3223) are greater than joining widths of the linear joining units at any position between adjacent ones of the intersecting positions.

### TECHNICAL FIELD

The present invention relates to a stretchable sheet structure having stretchability and to a disposable absorbent product having the stretchable sheet structure.

### CROSS-REFERENCE TO RELATED APPLICATIONS

This application claims priority benefit of Japanese Patent Applications No. JP2022-049269 filed in the Japan Patent Office on Mar. 25, 2022 and No. 2022-071690 filed in the Japan Patent Office on Apr. 25, 2022, the entire disclosures of which are incorporated herein by reference.

### BACKGROUND ART

Disposable absorbent products that receive body wastes from wearers are conventionally provided with gathers that have a stretchable sheet structure on various parts. In the case of a disposable diaper, for example, gathers are provided on parts such as those that come in contact with a groin, those that come in contact with around legs, and those that come in contact with around a waist. In order to prevent leakage, gathers are also provided on an auxiliary absorbent product arranged on the inside of the diaper.

As an example of such gathers, gathers provided with orderly ridges and grooves are known. For example, Japanese Utility Model Application Publication No. H4-71922 (Document 1) discloses a technique for forming gathers with a waist flap by arranging stretchable elastic members between nonwoven fabric sheets to which a hot-melt adhesive is applied linearly in fixed-interval strips, so that the stretchable elastic members intersect with line of the hot-melt adhesive.

Japanese Patent Application Laid-Open No. 2005-212405 (Document 2) discloses a technique in which when two sheet materials are intermittently heat-sealed in a stretching direction and a direction orthogonal to the stretching direction, elastic members facing the stretching direction are fixedly attached to the sheet materials by welding. International Publication No. 2018/070158 (Document 3) discloses a technique in which welding is utilized to join a pair of sheets together and to hold a plurality of elastic members between the pair of sheets.

As in Document 1, in the case where elastic members are fixedly attached with a hot-melt adhesive that is applied linearly in the direction perpendicular to the elastic member, a large amount of the hot-melt adhesive becomes necessary in order to properly join the elastic members. This enhances the property of the gathers trying to maintain their shape (hereinafter, referred to as “stiffness”) and degrades the touch of the gathers. As in Document 2, in the case where sheets and elastic members are joined together with dots of the same shape that are intermittently aligned in two dimensions, it is necessary to increase the width of the dots in a stretching direction in order to maintain joining strength. This reduces the area of sheet surfaces that can be used as ridges and grooves. According to Document 3, the elastic members are firmly caught at the positions of fixation during welding, but this may reduce the strength of the sheets at the positions of fixation of the elastic members.

Meanwhile, according to Documents 1 to 3, the ridges and grooves of the gathers are formed perpendicularly to the direction of extension of the gathers. This increases the stiffness of the gathers that tries to maintain their shape in the stretching direction of the ridges and grooves. As a result, for example, the gathers may too strongly come in contact with the groin, or the gathers around the waist may too strongly get caught on the skin surface when the diaper is worn.

### SUMMARY OF THE INVENTION

It is an object of the present invention to provide a stretchable sheet structure with appropriate stiffness while forming gathers with orderly ridges and grooves and to provide a disposable absorbent product having the stretchable sheet structure.

A first aspect of the present invention is a stretchable sheet structure that stretches in a stretching direction with a stretching force exerted thereon and shrinks when the stretching force is released. The stretchable sheet structure incudes a first sheet, a second sheet overlaid on the first sheet, a first elastic member positioned between the first sheet and the second sheet and extending linearly in the stretching direction, and a second elastic member positioned between the first sheet and the second sheet and extending linearly in the stretching direction while being spaced apart from the first elastic member. While the first sheet and the second sheet are stretched flat in the stretching direction, the first sheet and the second sheet are joined together by a plurality of linear joining units aligned in the stretching direction. Each linear joining unit intersects with the first elastic member and the second elastic member. A joining width of the linear joining unit at a first intersecting position of the linear joining unit and the first elastic member and a joining width of the linear joining unit at a second intersecting position of the linear joining unit and the second elastic member are greater than a joining width of the linear joining unit at any position between the first intersecting position and the second intersecting position. While the first elastic member and the second elastic member shrink, the first sheet or the second sheet has bulges between the plurality of linear joining units to form gathers.

According to the present invention, it is possible to provide the stretchable sheet structure with appropriate stiffness while forming gathers with orderly ridges and grooves.

A second aspect of the present invention is a stretchable sheet structure that stretches in a stretching direction with a stretching force exerted thereon and shrinks when the stretching force is released. The stretchable sheet structure includes a first sheet, a second sheet overlaid on the first sheet, a first elastic member positioned between the first sheet and the second sheet and extending linearly in the stretching direction, and a second elastic member positioned between the first sheet and the second sheet and extending linearly in the stretching direction while being spaced apart from the first elastic member. While the first sheet and the second sheet are stretched flat in the stretching direction, the first sheet and the second sheet are joined together by a plurality of linear joining units aligned in the stretching direction. Each of the plurality of linear joining units exists in an intersecting direction intersecting with the stretching direction, between the first elastic member and the second elastic member, the intersecting direction being inclined with respect to a direction perpendicular to the stretching direction. Each linear joining unit intersects with the first elastic member and the second elastic member. While the first elastic member and the second elastic member shrink, the first sheet or the second sheet has bulges between the plurality of linear joining units to form gathers.

A third aspect of the present invention is the stretchable sheet structure according to the second aspect, in which a joining width of the linear joining unit at a first intersecting position of the linear joining unit and the first elastic member and a joining width of the linear joining unit at a second intersecting position of the linear joining unit and the second elastic member are greater than a joining width of the linear joining unit at any position between the first intersecting position and the second intersecting position.

A fourth aspect of the present invention is the stretchable sheet structure according to the first or second aspect (or according to any one of the first to third aspects), in which each of the plurality of linear joining units continuously exists in an intersecting direction intersecting with the stretching direction, between the first elastic member and the second elastic member.

A fifth aspect of the present invention is the stretchable sheet structure according to the first or second aspect (or according to any one of the first to fourth aspects), in which the first sheet and the second sheet are joined together at each linear joining unit by welding.

A sixth aspect of the present invention is the stretchable sheet structure according to the first or second aspect (or according to any one of the first to fifth aspects), in which the first sheet and the second sheet are a single sheet folded along a folding line, while the first sheet and the second sheet are stretched flat in the stretching direction, the first elastic member is positioned in close proximity to and in parallel with the folding line, and each linear joining unit does not overlap with the folding line.

A seventh aspect of the present invention is the stretchable sheet structure according to the first or second aspect (or according to any one of the first to sixth aspects) that further includes a third elastic member positioned between the first sheet and the second sheet and extending linearly in the stretching direction while being spaced apart from the second elastic member on a side of the second elastic member opposite to the first elastic member. Each of the plurality of linear joining units exits in an intersecting direction intersecting with the stretching direction, between the second elastic member and the third elastic member. Each linear joining unit intersects with the third elastic member. The gathers are formed while the first elastic member, the second elastic member, and the third elastic member shrink. Each linear joining unit is linear while the first sheet and the second sheet are stretched flat in the stretching direction.

An eighth aspect of the present invention is the stretchable sheet structure according to the first or second aspect (or according to any one of the first to sixth aspects) that further includes a third elastic member positioned between the first sheet and the second sheet and extending linearly in the stretching direction while being spaced apart from the second elastic member on a side of the second elastic member opposite to the first elastic member. Each of the plurality of linear joining units exists in an intersecting direction intersecting with the stretching direction, between the second elastic member and the third elastic member. Each linear joining unit intersects with the third elastic member. The gathers are formed while the first elastic member, the second elastic member, and the third elastic member shrink. While the first sheet and the second sheet are stretched flat in the stretching direction, the linear joining unit is bent at a position of the second elastic member.

A ninth aspect of the present invention is the stretchable sheet structure according to the eighth aspect, in which an angular direction in which each linear joining unit is inclined with respect to the direction perpendicular to the stretching direction between the first elastic member and the second elastic member is opposite to an angular direction in which each linear joining unit is inclined with respect to the direction perpendicular to the stretching direction between the second elastic member and the third elastic member.

A tenth aspect of the present invention is the stretchable sheet structure according to the seventh aspect, in which while the first sheet and the second sheet are stretched flat in the stretching direction, the first sheet and the second sheet have an edge that exists along and in close proximity to the first elastic member on a side of the first elastic member opposite to the second elastic member, and an interval between the first elastic member and the second elastic member is greater than an interval between the second elastic member and the third elastic member.

An eleventh aspect of the present invention is the stretchable sheet structure according to the eighth aspect (or according to the eighth or ninth aspect), in which while the first sheet and the second sheet are stretched flat in the stretching direction, the first sheet and the second sheet have an edge that exists along and in close proximity to the first elastic member on a side of the first elastic member opposite to the second elastic member, and an interval between the first elastic member and the second elastic member is greater than an interval between the second elastic member and the third elastic member.

A twelfth aspect of the present invention is the stretchable sheet structure according to the first or second aspect (or according to any one of the first to eleventh aspects), in which while the first sheet and the second sheet are stretched flat in the stretch direction, a range of existence of each linear joining unit in the stretching direction overlaps in part with a range of existence of another linear joining unit in the stretching direction, the another linear joining unit being adjacent to the linear joining unit.

A thirteenth aspect of the present invention is a disposable absorbent product that receives a body waste from a wearer and includes the stretchable sheet structure according to the first or second aspect (or according to any one of the first to twelfth aspects).

A fourteenth aspect of the present invention is the disposable absorbent product according to the thirteenth aspect, in which the gathers come in contact with a groin of the wearer.

These and other objects, features, aspects and advantages of the present invention will become more apparent from the following detailed description of the present invention when taken in conjunction with the accompanying drawings.

### BRIEF DESCRIPTION OF DRAWINGS

FIG. 1 is a plan view showing a configuration of an absorbent product.

FIG. 2 is a perspective view showing one free state of the absorbent product.

FIG. 3 is a sectional view of the absorbent product.

FIG. 4 is a sectional view of the absorbent product.

FIG. 5 is a sectional view showing the vicinity of one gather sheet in enlarged dimensions.

FIG. 6 is a diagram showing the vicinity of a boundary between one central joining region and one end joining region in enlarged dimensions.

FIG. 7 is a plan view showing part of one gather sheet.

FIG. 8 is a plan view showing part of the absorbent product.

FIG. 9 is a diagram showing linear joining units in enlarged dimensions.

FIG. 10 is a perspective view showing bulges of inside and outside sheets.

FIG. 11 is a diagram showing another example of the linear joining units.

FIG. 12 is a diagram showing yet another example of the linear joining units.

FIG. 13 is a diagram showing yet another example of the linear joining units.

FIG. 14 is a diagram showing yet another example of the linear joining units.

FIG. 15 is a sectional view showing another example of the gather sheet that is standing.

FIG. 16 is a sectional view showing yet another example of the gather sheet that is standing.

### DESCRIPTION OF EMBODIMENTS

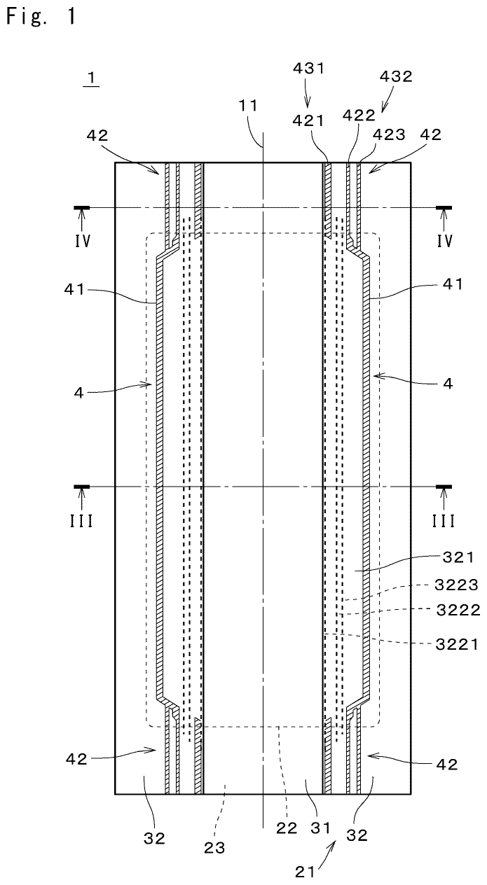

FIG. 1 is a plan view showing a configuration of a disposable absorbent product 1 (hereinafter, also simply referred to as the “absorbent product 1”) according to one embodiment of the present invention. FIG. 1 shows a state in which the absorbent product 1 is opened in a planar state. The absorbent product 1 is an auxiliary absorbent pad that is arranged on the inner side (i.e., wearer side) of an exterior product worn by a wearer to receive body wastes from the wearer. Examples of the exterior product include a disposable diaper, a non-disposable diaper, and a diaper-shaped cover. For example, the absorbent product 1 may be used for a person who gets care in a hospital or a nursing-care facility. In FIG. 1, the surface of the absorbent product 1 on the side that comes in contact with the wearer is shown toward the front. In the following description, the up-down direction in FIG. 1 is referred to as the “longitudinal direction,” and the right-left direction perpendicular to the longitudinal direction is referred to as the “width direction.” The absorbent product 1 is bilaterally symmetrical, and the center line that extends in the longitudinal direction in FIG. 1 is given a reference sign 11.

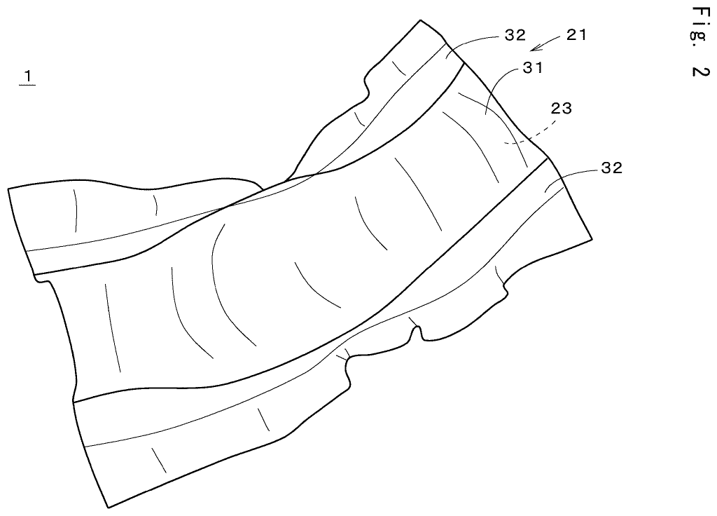

FIG. 2 is a perspective view showing one free state of the absorbent product 1. As will be described later, the absorbent product 1 includes elastic members and are curved as a whole due to shrinkage of the elastic members.

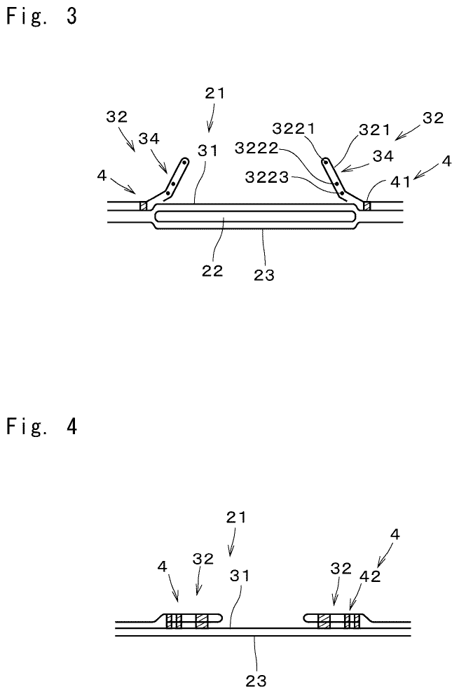

FIG. 3 is a sectional view of the absorbent product 1, taken along a plane perpendicular to the longitudinal direction at a position III-III in FIG. 1. Since gather sheets described later have gathers that stand up due to a shrinkage force of the elastic members as shown in FIG. 2, FIG. 3 shows a state in which the gathers are standing. FIG. 4 is a sectional view of the absorbent product 1, taken along a plane perpendicular to the longitudinal direction at a position IV-IV in FIG. 1.

As shown in FIGS. 1 to 4, the absorbent product 1 includes a top sheet 21, an absorbent core 22, and a back sheet 23. The absorbent core 22 is positioned between the top sheet 21 and the back sheet 23. The top sheet 21 includes a center sheet 31 and a pair of gather sheets 32. The pair of gather sheets 32 is fixedly attached on both side portions of the center sheet 31 (i.e., at spaced positions on both sides in the width direction) and extends in the longitudinal direction. Each gather sheet 32 exists along the overall longitudinal length of the center sheet 31. In other words, the longitudinal length of each gather sheet 32 is substantially the same as the longitudinal length of the absorbent product 1. Inner portions of the pair of gather sheets 32 in the width direction, i.e., gathers 34, stand up toward the wearer when the absorbent product 1 is worn.

In FIG. 1, an upper portion and a lower portion of the absorbent product 1 correspond respectively to a front part that comes in contact with the skin on the belly side of the wearer and a rear part that comes in contact with the skin on the back side of the wearer. A portion of the absorbent product 1 that is positioned between and continuous from the front part and the rear part is a central part that faces a groin of the wearer. There is no need to accurately determine boundaries between the front part and the central part and between the rear part and the central part. The front, central, and rear parts of the absorbent product 1 are arranged in this order in the longitudinal direction. In the example shown in FIG. 1, the absorbent product 1 has an approximately rectangular shape in plan view, and the front, central, and rear parts have substantially the same width in the width direction (i.e., the width in the right-left direction when the absorbent product 1 is worn; hereinafter also simply referred to as the “width”). In other words, the absorbent product 1 has an approximately constant width along its overall longitudinal length. FIGS. 3 and 4 described above are sectional views of the central part and the front part of the absorbent product 1, respectively. A section of the rear part of the absorbent product 1 is substantially similar to the section of the front part shown in FIG. 4. The width of the absorbent product 1 does not necessarily have to be constant, and for example, the width of the rear part may be greater than the width of the front part.

As shown in FIGS. 1, 3, and 4, the absorbent core 22 is sandwiched between the top sheet 21 and the back sheet 23. The top sheet 21 and the back sheet 23 are joined together around the absorbent core 22 with an adhesive. In FIG. 1, the contour of the absorbent core 22 is illustrated with a thin broken line. In FIGS. 3 and 4, to facilitate understanding of the drawings, the absorbent product 1 is illustrated greater than the actual dimension in the thickness direction (i.e., the up-down direction in FIGS. 3 and 4), and constituent elements of the absorbent product 1 that are in contact with each other or are joined together in reality are illustrated with appropriate spaces in the thickness direction.

The center sheet 31 of the top sheet 21 speedily captures moisture from body wastes received from the wearer and transmits the moisture to the absorbent core 22. The absorbent core 22 absorbs the moisture transmitted through the center sheet 31 and speedily immobilizes the moisture. The back sheet 23 prevents the moisture or the like in the body wastes most of which are transmitted through the center sheet 31 and the absorbent core 22 to the back sheet 23 from leaking to the outside of the absorbent product 1. In the example shown in FIG. 3, the absorbent core 22 is in contact with the center sheet 31 and the back sheet 23, but there may be a mount (e.g., tissue paper) arranged between the absorbent core 22 and the center sheet 31 or between the absorbent core 22 and the back sheet 23. The absorbent core 22 may be enclosed by any other sheet, or may be obtained by overlaying a plurality of absorbent cores one above another. That is, the absorbent core 22 and its peripheral structure may adopt any of various known structures.

The longitudinal lengths of the top sheet 21 and the back sheet 23 are greater than the length of the absorbent core 22. The widths of the top sheet 21 and the back sheet 23 in the width direction are greater than the width of the absorbent core 22. Both side portions of the top sheet 21 (i.e., portions on both sides in the width direction) are joined with both side portions of the back sheet 23 either directly or indirectly via an inclusion along the overall length. The width of the center sheet 31 may be smaller than the width of the back sheet 23. In this case, outer end portions of the gather sheet 32 in the width direction may be joined with the back sheet 23. In the longitudinal end portions of the absorbent product 1, the longitudinal end portions of the top sheet 21 are joined either directly or indirectly with the longitudinal end portions of the back sheet 23. The top sheet 21 and the back sheet 23 are joined together along the entire periphery of the absorbent core 22 and accordingly the absorbent core 22 as a whole is surrounded by the top sheet 21 and the back sheet 23. For example, the top sheet 21 and the back sheet 23 may be joined together with a hot-melt adhesive. The top sheet 21 and the back sheet 23 may be joined together by any of various methods other than using the hot-melt adhesive.

As the center sheet 31, for example, a liquid-permeable nonwoven fabric (e.g., a point-bonded nonwoven fabric, an air-through nonwoven fabric, or a spun-bonded nonwoven fabric) may be used that is formed of hydrophobic fiber (e.g., polypropylene, polyethylene, polyester, polyamide, or nylon) having a surface subjected to hydrophilic treatment using a surface-active agent. Note that the center sheet 31 may also be a nonwoven fabric (e.g., a spun lace nonwoven fabric) formed of hydrophilic fiber such as cellulose, rayon, or cotton.

Preferably, the back sheet 23 may have a layered structure in which a plurality of sheets are overlaid one above another. For example, the back sheet 23 may have a structure in which a nonwoven fabric is overlaid on a plastic film or a water-repellent or liquid-impermeable nonwoven fabric. In the case where the back sheet 23 includes a plastic film, the plastic film is arranged so as to face the absorbent core 22. Preferably, the plastic film may have moisture permeability (air permeability). This improves water resistance of the back sheet 23 and also improves the wearer's comfortability while preventing stuffiness inside the absorbent product 1. Examples of the non-woven fabric overlaid on the plastic film include water-repellent non-woven fabrics or liquid-impermeable nonwoven fabrics. This improves the feel of the back sheet 23 such as touch and texture. The nonwoven fabric may, for example, be a spun-bonded nonwoven fabric or a melt-blown nonwoven fabric, which are formed of hydrophobic fiber, or may be an SMS (spun-bonded, melt-blown, and spun-bonded) nonwoven fabric and may be subjected to water-repellent processing as necessary.

The absorbent core 22 is an approximately sheet-like member whose thickness is smaller than its length and width. The absorbent core 22 is provided across the front, central, and rear parts of the absorbent product 1. That is, the absorbent core 22 is positioned on the belly side, the inseam, and the back side of the wearer. The shape of the absorbent core 22 in plan view may be modified in various ways. For example, the width of the rear part of the absorbent core 22 may be greater than the width of the front part. Alternatively, the width of the central part of the absorbent core 22 may be smaller than the widths of the front and rear parts.

The absorbent core 22 may be configured by, for example, dispersing a high-absorbent material into a sheet fiber assembly. The fiber assembly may, for example, be an assembly of pulp fiber and/or synthetic fiber. The high-absorbent material may, for example, be a particulate SAP (super absorbent polymer) or a fibrous SAF (super absorbent fiber). The absorbent core 22 may also be a sheet fiber assembly that does not contain any high-absorbent material. As another alternative, the absorbent core 22 may be a substance that substantially does not contain any fiber such as pulp fiber and that contains a high-absorbent material fixedly attached between two liquid-permeable sheet members (e.g., nonwoven fabrics) with a hot-melt adhesive or the like.

Each gather sheet 32 includes a side sheet 321 and elastic members 3221 to 3223 as shown in FIGS. 1 to 3. In FIGS. 1 and 3, reference signs 321 and 3221 to 3223 are given to only the gather sheet 32 on the right side. The side sheet 321 is an appropriately strip sheet member that is long in the longitudinal direction. In the present embodiment, the gather sheets 32 on the right and left sides are each provided with the three elastic members 3221 to 3223. Each of the elastic members 3221 to 3223 has a linear shape extending in the longitudinal direction and is fixedly attached to an inner region of the side sheet 321 in the width direction. While the absorbent product 1 is spread flat as shown in FIG. 1, the elastic members 3221 to 3223 are stretched, and when the elastic members 3221 to 3223 shrink, the gather sheets 32 shrink and the absorbent product 1 is curved in an approximately cup shape as shown in FIG. 2. As a result, the inner portions of the gather sheets 32 in the width direction stand up from the center sheet 31 as shown in FIG. 3.

FIG. 5 is a diagram showing the vicinity of the gather sheet 32 on the right side in FIG. 3 in enlarged dimensions. The gather sheet 32 on the left side also has the same structure. As shown in FIG. 5, the side sheet 321 has a structure in which an inside sheet 331 and an outside sheet 332 are overlaid one above the other by folding a single sheet. The folding position is at an inner edge 333 of the gather sheet 32 in the width direction. The three elastic members 3221 to 3223 are arranged between the inside sheet 331 and the outside sheet 332. Hereinafter, the three elastic members 3221 to 3223 are also referred to as the “first elastic member 3221,” the “second elastic member 3222,” and the “third elastic member 3223” in order from the inner side to the outer side in the width direction. The first elastic member 3221 is arranged along and in the vicinity of the edge 333 of the gather sheet 32. The second and third elastic members 3222 and 3223 are arranged and spaced outward in the width direction from the first elastic member 3221.

As the side sheet 321, a water-repellent or liquid-impermeable nonwoven fabric formed of hydrophobic fiber (e.g., a spun-bonded nonwoven fabric, a melt-blown nonwoven fabric, or an SMS nonwoven fabric) may be used and may be subjected to water-repellent processing as necessary. As the elastic members 3221 to 3223, for example, polyurethane yarn, a strip polyurethane film, or filiform or strip natural rubber may be used.

The side sheet 321 is fixedly attached on the center sheet 31. In FIGS. 1, 3, and 4, regions in which the center sheet 31 and the side sheets 321 are joined together, i.e., regions in which the center sheet 31 and the gather sheets 32 are joined together, are cross-hatched. In the present embodiment, the center sheet 31 and the pair of gather sheets 32 are joined together by welding involving pressure. Hereinafter, these joining regions are referred to as a “compression joining region 4.” The term “welding involving pressure” as used herein refers to, for example, thermal welding in which the sheets are pressed by the application of heat or to ultrasonic welding in which the sheets are pressed by the application of ultrasonic vibrations, and is so-called “embossing.” Since the compression is caused by pressure, “welding involving pressure” can also be regarded as “welding involving compression.” Note that the joints between the center sheet 31 and the gather sheet 32 may be made by using a different joining direction such as a method using a hot-melt adhesive in combination.

The compression joining region 4 includes central joining regions 41 that are positioned in the range of a central portion in the longitudinal direction and end joining regions 42 that are positioned in the ranges of both end portions in the longitudinal direction. As shown in FIG. 1, two central joining regions 41 and four end joining regions 42 exist within one absorbent product 1. Each central joining region 41 is spaced outward in the width direction from the three elastic members 3221 to 3223. Each end joining region 42 does not overlap with the three elastic members 3221 to 3223. As shown in FIG. 1, each central joining region 41 is long and slender in the longitudinal direction. Each end joining region 42 includes three linear regions that extend in the longitudinal direction. The three linear regions are hereinafter referred to as the “first linear region 421,” the “second linear region 422,” and the “third linear region 423” in order from the inner side to the outer side in the width direction. In FIG. 1, the reference signs 421 to 423 are given to only the linear regions shown in the upper right portion. The second linear region 422 and the third linear region 423 exist outward of the second and third elastic members 3222 and 3223 in the width direction. Thus, these linear regions are collectively referred to as an “outer joining region 432,” and the first linear region 421 that exists inward of the second and third elastic members 3222 and 3223 in the width direction is also referred to as an “inner joining region 431.”

The inner joining region 431 and the outer joining region 432 are spaced from each other in the width direction. The positions of the second and third elastic members 3222 and 3223 in the width direction are located between the inner joining region 431 and the outer joining region 432. The position of the innermost first elastic member 3221 in the width direction is located inward of the inner joining region 431 in the width direction. That is, the positions in the width direction of the three elastic members 3221 to 3223 overlap with none of the ranges of existence of the inner joining region 431 and the outer joining region 432 in the width direction.

In the manufacture of the absorbent product 1, a plurality of absorbent cores 22 are sandwiched between the top sheet 21 and the back sheet 23 that are both continuous and correspond to a plurality of absorbent products 1, and thereafter an individual absorbent product 1 is cut out and obtained from this layered continuum. Since the three elastic members 3221 to 3223 do not overlap with the end joining regions 42, the end portions of the three elastic members 3221 to 3223 after cutting freely shrink within the gather sheet 32. Therefore, the end joining regions 42 do not receive any force from the elastic members 3221 to 3223 after cutting. As a result, it is possible to prevent the end joining regions 42 hardened by the compression joint from being inwardly curved significantly, i.e., toward the wearer side, upon receipt of the force from the elastic members 3221 to 3223 and thereby to improve the wearing comfort of the absorbent product 1. Although, in FIG. 1, the ranges of existence of the end joining regions 42 in the longitudinal direction overlap with the ranges of existence of the elastic members 3221 to 3223 in the longitudinal direction, theses ranges of existence may not overlap with one another.

The second linear region 422 and the third linear region 423 are connected to each other on the inner side in the longitudinal direction. By dividing the outer joining region 432 into the second linear region 422 and the third linear region 423, it is possible to improve the flexibility of the absorbent product 1 in the regions positioned outward of the front and rear parts of the absorbent product 1 in the width direction. As a result, the regions positioned outward of the front and rear parts in the width direction can easily come along the inner surface of the diaper (including a diaper-shaped cover; the same applies below) or the body of the wearer, and accordingly wearing comfort is improved. Besides, the air permeability of the end joining regions 42 is also improved.

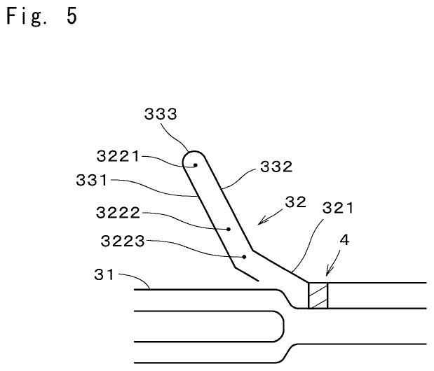

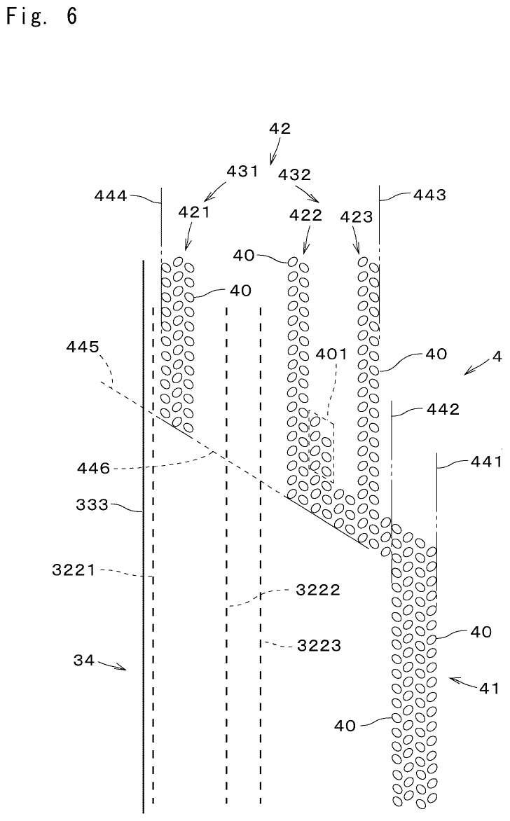

FIG. 6 is a diagram showing the vicinity of a boundary between one central joining region 41 and one end joining region 42 in the upper right portion of FIG. 1 in enlarged dimensions. In FIG. 6, a large number of dots 40 each represent one piece of the compression joining region 4, i.e., one dot-shaped embossment, and correspond to one recessed region. The dots 40 are hereinafter referred to as the “joining elements 40.” Although a large number of joining elements 40 are formed in the compression joining region 4, the intervals between the joining elements 40 are small and therefore a region that include spaces between the joining elements 40 is regarded as the compression joining region 4.

As indicated by a leader line in the upper portion in FIG. 6, an outer edge of the end joining region 42 in the width direction (at a position 443 in the width direction) is located in the width direction inward of the inner edge of the central joining region 41 in the width direction (at a position 442 in the width direction). In this way, by positioning the end joining region 42 inward of the central joining region 41 in the width direction, there are no portions hardened by the compression joints in the regions positioned outward of the front and rear parts of the absorbent product 1 in the width rection, and accordingly the flexibility is improved. As a result, the regions positioned outward of the front and rear parts in the width direction can easily come along the inner surface of the diaper or the body of the wearer, and accordingly, wearing comfort is improved. The central joining region 41 positioned outward of the end joining region 42 in the width direction allows the gathers 34 to stand up to a large degree and effectively prevents lateral leakage of the body wastes. Preferably, the dimension of the end joining region 42 in the width direction, i.e., the distance between the position 443 of the outer edge of the end joining region 42 and a position 444 of the inner edge of the end joining region 42, is greater than the dimension of the central joining region 41 in the width direction, i.e., the distance between a position 441 of the outer edge of the central joining region 41 and the position 442 of the inner edge thereof.

Here, the central joining region 41 and the outer joining region 432 are continuous in the longitudinal direction. This prevents the body wastes received by the absorbent product 1 from leaking out from the space between the central joining region 41 and the outer joining region 432. As described previously, the intervals of the joining elements 40 are narrow to such an extent that can prevent leakage of the body wastes. More preferably, the compression joining region 4 may exist along the overall longitudinal length of the top sheet 21. In this case, when the compression joining region 4 is formed using ultrasonic vibrations, the ultrasonic vibrations continuously act on the projections of a roll (so-called embossing roll) that form the compression joining region 4. As a result, the ultrasonic vibrations stably act on the projections of the embossing roll and reduces variations in the strength of the welding joints. Besides, the wearing away of the projections of the embossing roll is suppressed, and the life of the embossing roll is improved. When the compression joining region 4 is formed by thermal welding as well, it is possible to suppress variations in linear pressure generated between the pair of rolls that sandwich the sheet on which the compression joining region 4 is formed, and to reduce variations in the strength of the welding joints. Besides, the wearing away of the projections of the embossing roll is suppressed, and the life of the embossing roll is improved.

More preferably, a maximum value for the total length of the compression joints in the compression joining region 4 in the width direction is 1.25 times or more and 4 times or less of a minimum value therefor. Here, assuming a straight line extending in the width direction, the term “total length of the compression joints” as used herein refers to the total length of the ranges in which the compression joints are actually made on the straight line by the joining members 40 or the like (when there is only one range, the length of this range). More preferably, the maximum value for the total length of the compression joints may be 1.5 times or more and 3 times or less of the minimum value therefor. This allows the ultrasonic vibrations to stably act on the projections of the embossing roll and thereby reduces variations in the strength of the welding joints. Besides, the wearing away of the projections of the embossing roll is suppressed, and the life of the embossing roll is improved. In the case where the compression joining region 4 is formed by thermal welding as well, it is possible to suppress variations in linear pressure generated between the pair of rolls that sandwich the sheet on which the compression joining region 4 is formed, and thereby to reduce variations in the strength of the welding joint. Besides, the wearing away of the projections of the embossing roll is suppressed, and the life of the embossing roll is improved.

The edge of the end joining region 42 on the central side in the longitudinal direction (the line denoted by a reference sign 445 in FIG. 6; portions corresponding to the edge of the end joining region 42 are indicated with solid lines, and portions other than the edge are indicated with broken lines) is a discontinuous line that is interrupted at the positions of the second and third elastic members 3222 and 3223. The edge is inclined toward the center in the longitudinal direction as the edge approaches outward in the width direction. Note that the edge is not limited to a straight line and may be a curve. An additional joining region 401 exists outward of the range in which the edge (denoted by a reference sign 446 in FIG. 6) is interrupted in the width direction. This reduces variations in the range of the projections of the embossing roll on which the ultrasonic vibrations act during rotation of the embossing roll, and accordingly reduces variations in the strength of the welding joints. Besides, the wearing away of the projections of the embossing roll is suppressed, and the life of the embossing roll is improved. In the case where the compression joining region 4 is formed by thermal welding as well, it is possible to suppress variations in linear pressure generated between the pair of rolls and thereby to reduce variations in the strength of the welding joints. Besides, the wearing away of the projections of the embossing roll is suppressed, and the life of the embossing roll is improved. Note that the “additional joining region” can be grasped as, for example, distortion of the edge that originally ought to exist linearly or as a joining region that exists independently, from the shape around the end joining region 42. The additional joining region 401 may be provided inward of the interrupted range 446 of the edge in the width direction.

Now, for the sake of convenience, the edge of the central joining region 41 that corresponds to the outer position 441 of the outermost joining element 40 in the width direction in the central joining region 41 is referred to as the “outer edge 441,” the edge of the central joining region 41 that corresponds to the inner position 442 of the innermost joining element 40 in the width direction is referred to as the “inner edge 442,” the edge of the end joining region 42 that corresponds to the outer position 443 of the outermost joining element 40 in the width direction in the end joining region 42 is referred to as the “outer edge 443,” and the edge of the end joining region 42 that corresponds to the inner position 444 of the innermost joining element 40 in the width direction is referred to as the “inner edge 444.” The ratio of existence of the joining elements 40 between the outer edge 443 and the inner edge 444 of the end joining region 42 is lower than the ratio of existence of the joining elements 40 between the outer edge 441 and the inner edge 442 of the central joining region 41. The term “ratio of existence of the joining elements 40” as used herein refers to the ratio of the number of joining elements 40 or the total length of the range of existence of the joining elements 40 to the width of the compression joining region 4 when the compression joining region 4 is cut in a straight line extending in the width direction. Note that the “ratio of existence of the joining elements 40” may be the ratio of the area of the joining elements 40 to the area of a target region as a whole.

Since the ratio of existence of the joining elements 40 in the end joining region 42 is lower than the ratio of existence of the joining elements 40 in the central joining region 41, the joining elements 40 are non-densely dispersed in the end joining region 42. This improves the flexibility of the absorbent product 1 in the regions of the front and rear parts of the absorbent product 1 and, in particular, the regions positioned outward of the front and rear parts in the width direction. As a result, the wearing comfort of the regions of the front and rear parts is improved. Meanwhile, in the central joining region 41, the gather sheet 32 can be firmly and fixedly attached to the center sheet 31. This prevents lateral leakage of the body wastes.

As shown in FIG. 6, the first elastic member 3221 located at the innermost position in the width direction is positioned inward of the inner joining region 431 (first linear region 421) in the width direction. This prevents an unnecessary force caused by the first elastic member 3221 from acting on the inside of the first linear region 421 in the width direction while forming ridges and grooves up to the inner edge 333 of the gather sheet 32.

A portion of the gather sheet 32 that is positioned inward of the compression joining region 4 in the width direction is not fixedly attached on the center sheet 31. This portion of the gather sheet 32 can be spaced above from the center sheet 31 (i.e., toward the wearer side when the absorbent product 1 is worn). As shown in FIG. 5, the first elastic member 3221 is joined to the vicinity of the inner edge 333 of each side sheet 321 while being stretched and extending in the longitudinal direction. The second and third elastic members 3222 and 3223 are joined to the side sheet while being stretched and extending in the longitudinal direction and being spaced outward in the width direction from the first elastic member 3221. The second elastic member 3222 and the third elastic member 3223 are also spaced apart from each other in the width direction. When the absorbent product 1 is worn, the portion of each gather sheet 32 positioned inward of the central joining region 41 in the width direction is spaced above from the center sheet 31 and stands up toward the wearer side due to the shrinkage of the elastic members 3221 to 3223 as shown in FIG. 3. The shrinkage of the elastic members 3221 to 3223 produces the gathers 34 in the portion of the gather sheet 32 that is positioned inward of the central joining region 41 in the width direction. This allows the absorbent product 1 to prevent leakage of urine or the like from around the legs. Note that, as will be described later, the side sheet 321 and the elastic members 3221 to 3223 are joined together by welding using heat or ultrasonic vibrations, but they may be joined together with an adhesive.

The absorbent product 1 may be modified in various ways. For example, the back sheet 23 is not limited to a single sheet and may be configured by overlaying a plurality of sheets one above another, or may be a sheet obtained by joining a plurality of sheets together in the longitudinal or width direction. The back sheet 23 may have a function of preventing leakage of a liquid in the absorbent core 22 to the outside of the absorbent product 1. Typically, the back sheet 23 may include a plastic film provided with an infinite number of fine holes, and a nonwoven fabric joined to the outer surface of the plastic film. The expression for the “back sheet 23” means a sheet that covers the lower surface of the absorbent core 22, i.e., the surface on the side opposite to the wearer.

The expression for the “top sheet 21” means a sheet that covers the upper surface of the absorbent core 22, i.e., the surface facing the wearer. While the top sheet 21 of the disposable absorbent product 1 according to the present embodiment includes the center sheet 31 and the gather sheets 32, the top sheet 21 may further include different sheets other than those described above. The center sheet 31 has liquid permeability. However, there is no need for the center sheet 31 as a whole to have liquid permeability. The center sheet 31 is also not limited to a single sheet, and may be configured by overlaying a plurality of sheets one above another, or may be a sheet obtained by joining a plurality of sheets together in the longitudinal or width direction.

The gather sheets 32 mean the sheets including the gathers 34 that stand up toward the groin of the wearer, and the structure and shape of the gather sheets 32 are not limited to the examples described above. The gather sheets 32 may be joined to the lower surface of the center sheet 31 by welding involving pressure. In this case, for example, the gather sheets 32 may be joined to the lower surface of the center sheet 31 in the central joining region 41, and the portions of the gather sheets 32 that are folded upward at the edges of the center sheet 31 may be joined to the upper surface of the center sheet 31 in the end joining regions 42.

The side sheet 321 of each gather sheet 32 is not limited to the sheet configured by folding a single sheet and may be configured by overlaying a plurality of sheets one above another, or may be a sheet obtained by joining a plurality of sheets together in the longitudinal or width direction. The gather sheets 32 prevent leakage of the body wastes to the outside in the width direction, and at least the insides of the gather sheets 32 have hydrophobicity. Although the gather sheets 32 in FIG. 3 stand up from the outside toward the inside in the width direction, they may be folded outward in the width direction in the central joining region 41 and then stand up toward the groin of the wearer.

The number of elastic members included in each gather sheet 32 may be one, may be two, or may be four or more. That is, each gather sheet 32 is provided with at least one elastic member. Preferably, the elastic member(s) may be linear. The expression “linear” includes meaning of “thin filiform,” “flat-sectioned filiform,” and “flat-sectioned tape.” In any case, it is preferable that at least one elastic member does not overlap with the end joining regions 42. To be more precise, the position of at least one elastic member in the width direction does not overlap with the range of existence of the end joining regions 42 in the width direction. This reduces the possibility that the shrinkage force of at least one elastic member may act on the end joining regions 42. Preferably, one elastic member may be arranged along and in the vicinity of the inner edge 333 of the side sheet 321 in the width direction.

In the case where the number of elastic members is one, the outer joining region 432 is a region that exists outward of the elastic member in the width direction. The inner joining region 431 is a region that exists inward of the elastic member in the width direction. In the case where the number of elastic members is two or more, the outer joining region 432 is a region that exists outward of any of the elastic members in the width direction. The inner joining region 431 is a region that exists inward of the any of the elastic members in the width direction. To be expressed more generally, the outer joining region 432 is a region that exists outward of any elastic member among at least one elastic member (where the number of the any elastic member may be one or more) in the width direction, and the inner joining region 431 is a region that exists inward of the any elastic member in the width direction. Since the end joining regions 42 do not overlap with any of the elastic members, the end joining regions 42 will not receive any force directly from the elastic members. This prevents the end joining regions 42 hardened by the compression joints from being inwardly curved significantly (i.e., toward the viewer of the drawing in FIG. 1), i.e., toward the wearer, and accordingly improves the wearing comfort of the absorbent product 1. In other words, it is possible to reduce wearing discomfort when the absorbent product 1 is worn, as compared to the case where the end joining regions 42 overlap with the elastic members.

The presence of the inner joining region 431 and the outer joining region 432 and the presence of one or more (preferably, two as in the above-described embodiment) elastic members arranged in the width direction between the inner joining region 431 and the outer joining region 432 allows the portion of each gather sheet 32 in which the gathers 34 are formed to stand up easily. The presence of at least one elastic member between the inner joining region 431 and the outer joining region 432 stabilizes the overall shape of the gathers 34. In the gather sheet 32, in order to allow the ends of the gathers 34 to stand up with stability, it is preferable that the elastic member positioned at the innermost position in the width direction among a plurality of elastic members is positioned inward of the inner joining region 431 in the width direction. Moreover, in order to position the inner joining region 431 as far inward as possible, it is preferable that the elastic member positioned at the second innermost position from the inside in the width direction among the plurality of elastic members may be positioned between the inner joining region 431 and the outer joining region 432 in the width direction. That is, it is preferable that the number of elastic members positioned inward of the inner joining region 431 in the width direction may be one.

At least one elastic member may be provided in various ways on the gather sheet 32, and in any case, the range(s) of existence of the elastic member(s) in the longitudinal direction may or may not overlap with the range of existence of the end joining region 42 in the longitudinal direction.

In the above-described embodiment, the compression joining region 4 corresponds to a large number of dot-shaped joining elements 40, but it may also be a large number of linear joining elements or may have a mesh joining shape. Of course, the center sheet 31 and each gather sheet 32 may be welded together without any clearance across the compression joining region 4. In any case, it is preferable that a maximum value for the total length of the compression joints in the width direction in the compression joining region 4 may be 1.25 times or more and 4 times or less of the minimum value therefor. More preferably, the above maximum value for the total length may be 1.5 times or more and 3 times or less of the minimum value therefor.

Preferably, the compression joining region 4 may exist continuously from the front joining region 42 to the rear joining region 42. More preferably, the compression joining region 4 may exist along the overall length of the top sheet 21 in the longitudinal direction. The expression that the compression joining region “exists continuously” as used herein includes the case where the joining elements 40 are spaced apart from one another to such a degree that does not cause leakage of the body wastes. Moreover, the compression joining region 4 does not always have to exist continuously, and the center sheet 31 and the gather sheet 32 may be joined together with an adhesive at positions where the compression joining region 4 does not exist. In the above-described embodiment, each end joining region 42 is divided into the inner joining region 431 and the outer joining region 432, but the inner joining region 431 may be omitted. In this case, the outer joining region 432 corresponds with the end joining region 42, and it is preferable that the outer joining region 432 and the central joining region 41 may be continuous in the longitudinal direction, and accordingly, the compression joining region 4 as a whole is continuous in the longitudinal direction.

In the above-described embodiment, the outer edge 443 of each end joining region 42 in the width direction is positioned, in the width direction, inward of the inner edge 442 of the central joining region 41 in the width direction, but the outer edge 443 of the end joining region 42 in the width direction may be positioned, in the width direction, inward of only the outer edge 441 of the central joining region 41 in the width direction. In this case as well, it is possible to improve the flexibility of the outer portion(s) of the front and/or rear parts of the absorbent product 1 in the width direction. Since there is a case where the compression joining region 4 is an assembly of dot-shaped joining elements 40, the edges 441 to 444 mean, as described above, lines that are in contact with the inner and outer sides of the compression joining region 4. In the case where the edges 441 to 444 are curves, the language saying that one edge is positioned inward of another edge in the width direction means in principle that the one edge as a whole is positioned inward of the other edge as a whole in the width direction, but it also includes the case where an extremely small portion of the one edge or an extremely small portion of the other edge does not satisfy the above definition.

In the above-described embodiment, the outer joining region 432 includes the second linear region 422 and the third linear region 423 that extend in the longitudinal direction, but the outer joining region 432 may be configured by three or more linear regions. These linear regions do not necessarily have to be connected to one another. In this case, the outermost linear region may be regarded as the outer joining region 432. Forming the end joining region 42 as an assembly of linear regions improves the flexibility of the end joining region 42 and improves air permeability in the end joining region 42. It is preferable that at least a portion of the end joining region 42 on the end side in the longitudinal direction may be an assembly of a plurality of linear regions extending in the longitudinal direction.

Preferably, the center sheet 31 and the pair of gather sheets 32 may be joined together only in the compression joining region 4. That is, they may be joined together by only welding, and there is no adhesive applied therebetween. Of course, an adhesive may be used in an auxiliary manner as necessary when the center sheet 31 and the pair of gather sheets 32 are joined together.

The above-described absorbent product 1 is merely an example, and the joint made by welding between the center sheet 31 and the gather sheets 32 described in the above embodiment may be utilized as a pants-type disposable diaper, an open-type disposable diaper that is set to around the waist with tapes, an auxiliary absorbent pad arranged in a diaper or a diaper-shaped cover, a urine pad, or any of other various absorbent products that receive body wastes from a wearer.

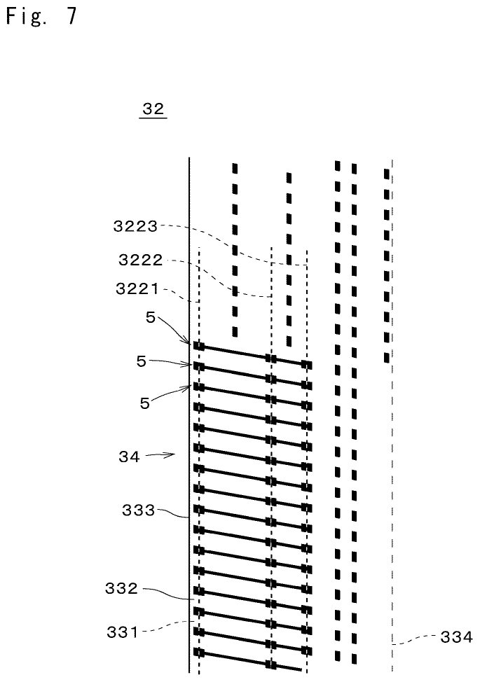

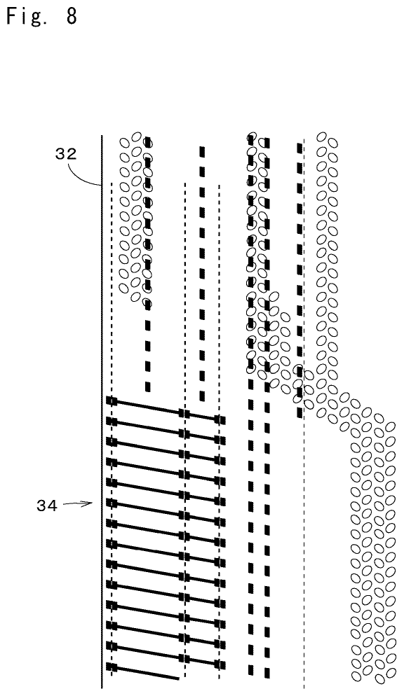

Next, the structure of each gather sheet 32 will be described. FIG. 7 is a plan view showing part of the gather sheet 32 on the right side in the front part of the absorbent product 1 shown in FIG. 1. FIG. 7 also shows the structure of the gather sheet 32, which is not shown in FIG. 6. FIG. 8 shows the structure of FIG. 7 in a portion of the absorbent product 1 that is similar to the portion shown in FIG. 6. FIG. 7 shows part of the portion of the gather sheet 32 in which the gathers 34 are formed, and part of an end portion in which the gathers 34 are not formed. The gathers 34 of the gather sheet 32 have a stretchable sheet structure that stretches in a stretching direction with a stretching force exerted thereon and shrinks when the stretching force is released.

In FIG. 7, thick lines that connect portions filled in with black and regions filled in with black indicate regions in which the inside sheet 331 and the outside sheet 332 (see FIG. 5) are joined together by welding involving pressure. A broken line 334 indicates the outer edge of the inside sheet 331 in the width direction. The regions joined by welding are so-called embossed regions. The term “welding involving pressure” refers to, for example, thermal welding in which the sheets are pressed by the application of heat or to ultrasonic welding in which the sheets area pressed by the application of ultrasonic vibrations, and is so-called “embossing.” Since the sheets are compressed by pressure, the term “welding involving pressure” can also be regarded as “welding involving compression.” Note that the joints between the inside sheet 331 and the outside sheet 332 may be made by a different joining direction such as a method using a hot-melt adhesive.

When the inside sheet 331 and the outside sheet 332 are generally referred to as a “first sheet” and a “second sheet,” respectively, the second sheet is overlaid on the first sheet as shown in FIG. 5. The first elastic member 3221 is an elastic member positioned between the first sheet and the second sheet and extending linearly in the stretching direction. The second elastic member 3222 is an elastic member positioned between the first sheet and the second sheet and extending linearly in the stretching direction while being spaced apart from the first elastic member 3221. The third elastic member 3223 is an elastic member positioned between the first sheet and the second sheet and extending linearly in the stretching direction while being spaced apart from the second elastic member 3222 on the side of the second elastic member 3222 opposite to the first elastic member 3221.

In FIG. 7, the up-down direction corresponds to the stretching direction of the gather sheet 32. While the gather sheet 32 is stretched flat in the stretching direction, i.e., while the inside sheet 331 and the outside sheet 332 are stretched flat in the stretching direction, the inside sheet 331 and the outside sheet 332 are joined together in the region of the gathers 34 by a plurality of linear joining units 5 aligned in the stretching direction. This produces the beautiful gathers 34 with orderly ridges and grooves.

In the case of the example in FIG. 7, the inside sheet 331 and the outside sheet 332 are joined together by welding in each linear joining unit 5, which is indicated as a region filled in with black. In the case of welding, the gathers 34 can be formed more beautifully as compared to using adhesive because it is possible to easily reduce the joining width of the linear joining units 5, in particular, the joining width at a position between the intersecting positions of a pair of elastic members. In the case where the joints are made by welding, it is preferable that an adhesive is not used in combination. That is, it is preferable that the joints are made by only welding.

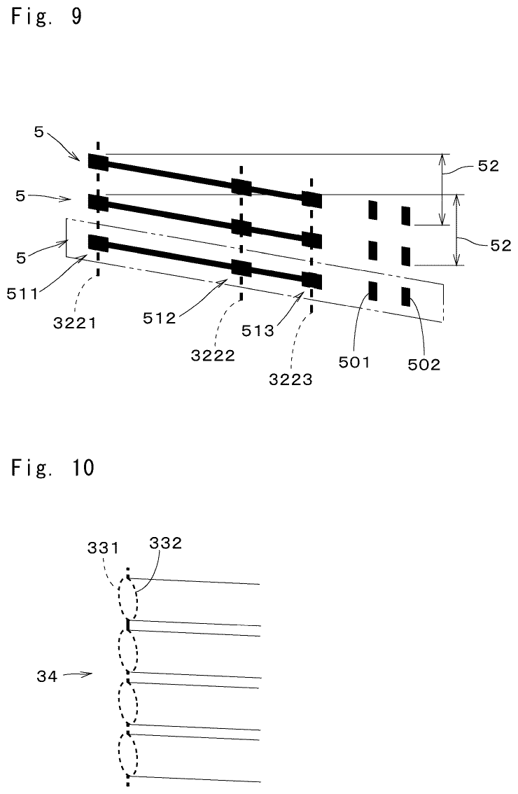

FIG. 9 is a diagram showing three linear joining units 5 in enlarged dimensions. In FIG. 9, one of the linear joining units 5 is enclosed by a chain double-dashed line. While the first elastic member 3221, the second elastic member 3222, and the third elastic member 3223 shrink, the inside sheet 331 or the outside sheet 332 has bulges between the plurality of linear joining units 5 to form the gathers 34. Of course, the inside sheet 331 and the outside sheet 332 both may have bulges between the plurality of linear joining units 5 to form the gathers 34. FIG. 10 is a perspective view showing a state in which the inside sheet 331 and the outside sheet 332 have bulges. Broken lines indicate sections of the gathers 34 that are cut along a plane perpendicular to the width direction. As shown in FIG. 10, the gather sheet 32 has the gathers 34 with orderly ridges and grooves.

As shown in FIG. 9, each of the plurality of linear joining units 5 exists continuously in a direction that intersects with the stretching direction between the first elastic member 3221 and the second elastic member 3222. Each of the linear joining units 5 also exists continuously in a direction that intersects with the stretching direction between the second elastic member 3222 and the third elastic member 3223. Continuous existence of the linear joining units 5 allows the formation of the beautiful gathers 34.

In the case of the example shown in FIG. 7, the direction of extension of the linear joining units 5 between the first elastic member 3221 and the second elastic member 3222 (i.e., the aforementioned “direction that intersects” between the first elastic member 3221 and the second elastic member 3222) is the same as the direction of extension of the linear joining units 5 between the second elastic member 3222 and the third elastic member 3223 (i.e., the aforementioned “direction that intersects” between the second elastic member 3222 and the third elastic member 3223). That is, each linear joining unit 5 is linear while the inside sheet 331 and the outside sheet 332 are stretched flat in the stretching direction. The aforementioned direction of intersection in which the linear joining units 5 exist continuously between the first elastic member 3221 and the third elastic member 3223 is hereinafter referred to as the “linear joining direction.” Since the linear joining units 5 are linear, it is possible to make linear the ridges and grooves extending from the first elastic member 3221 to the third elastic member 3223 and to form the beautiful gathers 34.

The linear joining direction is inclined with respect to the direction perpendicular to the stretching direction. All of the linear joining units 5 have the same linear joining direction. By setting the linear joining directions in such a manner, it is possible to weaken the force of the gathers 34 that is exerted on the skin. In particular, it is possible to weaken the instantaneous force of the gathers 34 that is exerted on the skin and to improve the feel when worn.

In the example shown in FIG. 9, two independent joining regions 501 and 502 exist on the side of the third elastic member 3223 opposite to the first elastic member 3221. These joining regions 501 and 502 may not be included in the linear joining units 5, or may be included in the linear joining units 5. In the case where the joining regions 501 and 502 are included in the linear joining units 5, the linear joining units 5 exist discontinuously on the side of the third elastic member 3223 opposite to the first elastic member 3221.

As shown in FIG. 5, the inside sheet 331 and the outside sheet 332 are a single sheet folded back at the edge 333. Since the edge 333 is the folding line of the single sheet, the edge 333 is hereinafter also referred to as the “folding line 333.” While the inside sheet 331 and the outside sheet 332 are stretched flat in the stretching direction as shown in FIG. 7, the first elastic member 3221 is positioned in close proximity to and in parallel with the folding line 333. Meanwhile, each linear joining unit 5 does not overlap with the folding line 333. That is, each linear joining unit 5 exists just before reaching the folding line (edge) 333. In other words, each linear joining unit 5 is spaced from the folding line 333. This prevents the gather sheet 32 from becoming hardened on the folding line (edge) 333 by welding and improves the touch of the gathers 34. In particular, it is possible to improve the feel when the ends of the gathers 34 come in contact with the groin. The distance between the folding line 333 and the first elastic member 3221 may preferably be less than or equal to 5 mm and more preferably less than or equal to 3 mm.

Each linear joining unit 5 intersects with the first elastic member 3221, the second elastic member 3222, and the third elastic member 3223. Here, the position of intersection between each linear joining unit 5 and the first elastic member 3221 is referred to as the “first intersecting position 511,” the position of intersection between each linear joining unit 5 and the second elastic member 3222 is referred to as the “second intersecting position 512,” and the position of intersection between each linear joining unit 5 and the third elastic member 3223 is referred to as the “third intersecting position 513” with reference to FIG. 9. The joining width of each linear joining unit 5 at the first intersecting position 511 is greater than the joining width of the linear joining unit 5 at any position between the first intersecting position 511 and the second intersecting position 512. The joining width of each linear joining unit 5 at the second intersecting position 512 is greater than the joining width of the linear joining unit 5 at any position between the first intersecting position 511 and the second intersecting position 512 and is also greater than the joining width of the linear joining unit 5 at any position between the second intersecting position 512 and the third intersecting position 513. The joining width of each linear joining unit 5 at the third intersecting position 513 is greater than the joining width of the linear joining unit 5 at any position between the second intersecting position 512 and the third intersecting position 513.

The term “joining width” may be defined variously as long as the width of each linear joining units 5 is approximately determined. For example, the joining width may be defined as the width (length) of the linear joining unit 5 that is cut along a straight line parallel to the stretching direction. The joining width may also be defined as the width (length) of the linear joining unit 5 that is cut along a straight line perpendicular to the linear joining direction. The joining width at a position between any adjacent two of the intersecting positions is smaller than the joining widths at the intersecting positions. In other words, the joining widths of the linear joining units 5 at the intersecting positions are relatively large.

If the joining widths at positions other than the intersecting positions are reduced relatively, the gathers 34 have large ridges and grooves formed at regular intervals (as compared to the case where the linear joining unit 5 as a whole is thickened). Meanwhile, at the intersecting positions, the first elastic member 3221, the second elastic member 3222, and the third elastic member 3223 can be firmly and fixedly attached to the inside sheet 331 and the outside sheet 332.

To be more strict, at the intersecting positions, the elastic members are fixedly attached to the inside sheet 331 and the outside sheet 332 while not being cut by heat. To achieve such fixation, the inside sheet 331 and the outside sheet 332 are welded together on both sides of the elastic members in the width direction, whereas the inside sheet 331 and the elastic members are only slightly welded together, and the outside sheet 332 and the elastic members are also only slightly welded together. In order to achieve such welding, the projections of the embossing roll, which are used during welding, have grooves formed therein to avoid the elastic members. Accordingly, for example, the inside sheet 331 and the elastic members are slightly welded together and the outside sheet 332 and the elastic member are slightly welded together on both sides of the elastic members in the width direction. Alternatively, while the elastic members are sandwiched between the inside sheet 331 and the outside sheet 332 under weak pressure, heat may be applied so that the elastic members are welded together with at least one of the inside sheet 331 and the outside sheet 332.

In the gathers 34, each linear joining unit 5 is inclined with respect to the direction perpendicular to the stretching direction between the first elastic member 3221 and the second elastic member 3222 and between the second elastic member 3222 and the third elastic member 3223. This allows the gathers 34 to become easily distorted when coming in contact with the skin. As a result, it is possible to improve the wearing comfort of the absorbent product 1.

In the example shown in FIG. 7, while the inside sheet 331 and the outside sheet 332 are stretched flat in the stretching direction, the edge 333 of the inside sheet 331 and the outside sheet 332 exists along and in close proximity to the first elastic member 3221 on the side of the first elastic member 3221 opposite to the second elastic member 3222. That is, the edge 333 of the gather sheet 32, the first elastic member 3221, the second elastic member 3222, and the third elastic member 3223 are positioned in this order toward the outside in the width direction. The interval between the first elastic member 3221 and the second elastic member 3222 is greater than the interval between the second elastic member 3222 and the third elastic member 3223. This allows the gathers 34 to become easily distorted on the end side of the gathers 34, i.e., on the side of the first elastic member 3221, and improves the waring comfort. This also allows the gathers 34 to have increased stiffness on the root side of the gathers 34, i.e., on the side of the third elastic member 3223 and thereby allows the gathers 34 to stand up with stability (the same also applies to FIGS. 11 to 14 and other variations described later).

As indicated by reference signs 52 in FIG. 9, while the inside sheet 331 and the outside sheet 332 are stretched flat in the stretching direction, an existence range 52 of each linear joining unit 5 in the stretching direction overlaps in part with an existence range 52 of a linear joining unit 5 adjacent to the above linear joining unit 5 in the stretching direction (the same also applies to FIGS. 11 to 14 and other variations described later). Even in the case where the linear joining units 5 are assumed to exist only between the first elastic member 3221 and the third elastic member 3223, the aforementioned existence ranges of adjacent ones of the linear joining units 5 overlap each other. This suppresses variations in linear pressure caused in the embossing roll during welding using the embossing roll and accordingly reduces variations in the strength of the welding joints and the occurrence of perforation during welding. Besides, the wearing away of the projections of the embossing roll is suppressed, and the life of the embossing roll is improved.

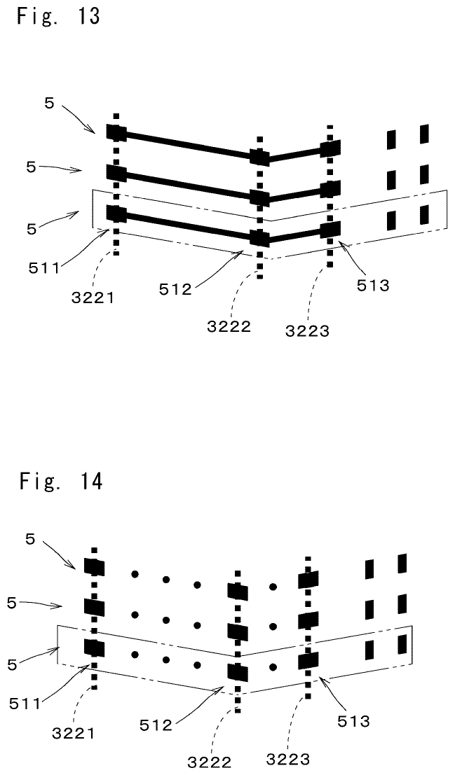

FIG. 11 is a diagram showing another example of the linear joining units 5 and shows three linear joining units 5 as in FIG. 9. The linear joining units 5 in FIG. 11 are discontinuous between the first elastic member 3221 and the second elastic member 3222 and are also discontinuous between the second elastic member 3222 and the third elastic member 3223. In this way, the linear joining units 5 do not necessarily have to be continuous between the elastic members. The other structure of the linear joining units 5 in FIG. 11 is similar to that shown in FIG. 9. That is, the joining width of each linear joining unit 5 at the first intersecting position 511 is greater than the joining width of the linear joining unit 5 at any position between the first intersecting position 511 and the second intersecting position 512. The joining width of each linear joining unit 5 at the second intersecting position 512 is greater than the joining width of the linear joining unit 5 at any position between the first intersecting position 511 and the second intersecting position 51, and is also greater than the joining width of the linear joining unit 5 at any position between the second intersecting position 512 and the third intersecting position 513. The joining width of each linear joining unit 5 at the third intersecting position 513 is greater than the joining width of the linear joining unit 5 at any position between the second intersecting position 512 and the third intersecting position 513. The same also applies to FIGS. 13 and 14 described below.

The linear joining units 5 are linear. In the gathers 34, each linear joining unit 5 is inclined with respect to the direction perpendicular to the stretching direction between the first elastic member 3221 and the second elastic member 3222 and between the second elastic member 3222 and the third elastic member 3223. The description of the linear joining units 5 in FIG. 9 is also applicable to the example shown in FIG. 11, except that the linear joining units 5 are continuous between the first elastic member 3221 and the third elastic member 3223.

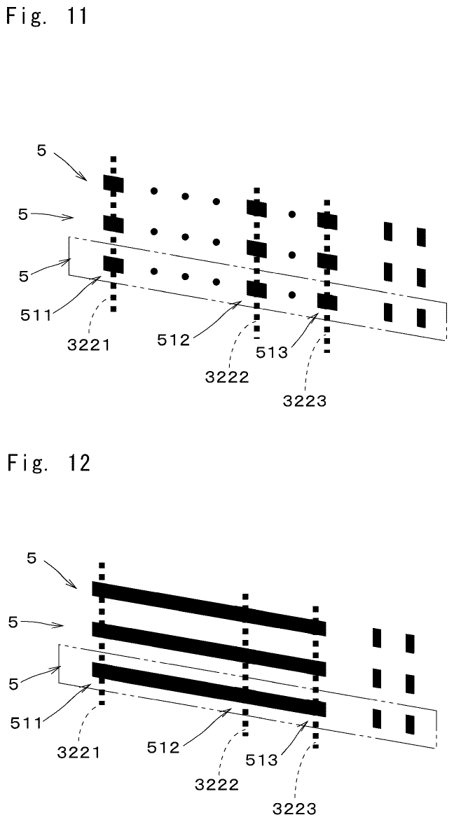

FIG. 12 is a diagram showing yet another example of the linear joining units 5 and shows three linear joining units 5 as in FIG. 9. The linear joining units 5 in FIG. 12 have a constant joining width between the first elastic member 3221 and the third elastic member 3223. That is, the joining widths at the first intersecting position 511, the second intersecting position 512, and the third intersecting position 513 are the same as the joining width at any position between the first intersecting position 511 and the second intersecting position 512 and are also the same as the joining width at any position between the third intersecting position 513 and the second intersecting position 512. The linear joining units 5 may have a constant joining width as long as the intervals of the linear joining units 5 are great enough to form sufficiently large ridges and grooves. The description of the linear joining units 5 in FIG. 9 is also applicable to the example shown in FIG. 12, except that the linear joining units 5 have a constant joining width.

FIG. 13 is a diagram showing yet another example of the linear joining units 5 and shows three linear joining units 5 as in FIG. 9. While the inside sheet 331 and the outside sheet 332 are stretched flat in the stretching direction, each linear joining unit 5 in FIG. 13 is bent at the position of the second elastic member 3222, i.e., at the second intersecting position 512. The other structure of the linear joining units 5 in FIG. 13 is similar to that of the linear joining units 5 shown in FIG. 9, and the description of the linear joining units 5 in FIG. 9 is also applicable to the example in FIG. 13, except that the linear joining units 5 are bent.

Each of the linear joining units 5 in FIG. 13 exists in a first intersecting direction that intersects with the stretching direction between the first elastic member 3221 and the second elastic member 3222 and exists in a second intersecting direction that intersects with the stretching direction between the second elastic member 3222 and the third elastic member 3223. Since the first intersecting direction and the second intersecting direction are different directions, the linear joining units 5 are bent. Each linear joining unit 5 intersects with the first elastic member 3221, the second elastic member 3222, and the third elastic member 3223 at the first intersecting position 511, the second intersecting position 512, and the third intersecting position 513, respectively. In the gathers 34, while the first elastic member 3221, the second elastic member 3222 and the third elastic member 3223 shrink, ridges and grooves are formed that are bent between each pair of adjacent linear joining units 5. The gathers 34 are formed as an assembly of these ridges and grooves.

The ridges and grooves that are bent can reduce the stiffness of the gathers 34. That is, the gathers 34 become easily deformed at the second intersecting position 512 when the gather sheets 32 shrink to bring the edge 333 (see FIG. 5) in abutment with the body. This allows the gathers 34 to smoothly come in contact with the body and accordingly improves the wearing comfort of the absorbent product 1. Besides, a large internal space can be ensured within the gathers 34 by bending the gathers 34. Therefore, it is possible to receive a large amount of body wastes at once.

Preferably, the first intersecting direction and the second intersecting direction described above may be opposite directions with respect to the direction perpendicular to the stretching direction. In other words, an angular direction in which each linear joining unit 5 is inclined with respect to the direction perpendicular to the stretching direction between the first elastic member 3221 and the second elastic member 3222 is opposite to an angular direction in which each linear joining unit 5 is inclined with respect to the direction perpendicular to the stretching direction between the second elastic member 3222 and the third elastic member 3223. In yet other words, each linear joining unit 5 has an approximately V-shape. This allows each ridge and groove to be bent like a spring and accordingly improves the touch of the gathers 34.

In order to allow the gathers 34 to stand up more properly, the direction of extension of the linear joining units 5, i.e., the second intersecting direction, may be perpendicular to the stretching direction between the second elastic member 3222 and the third elastic member 3223.

FIG. 14 is a diagram showing yet another example of the linear joining unit 5 and shows three linear joining units 5 as in FIG. 13. While the inside sheet 331 and the outside sheet 332 are stretched flat in the stretching direction, each linear joining unit 5 in FIG. 14 is bent at the position of the second elastic member 3222, i.e., at the second intersecting position 512. The linear joining units 5 in FIG. 14 are discontinuous between the first elastic member 3221 and the second elastic member 3222 and are also discontinuous between the second elastic member 3222 and the third elastic member 3223. The other structure of the linear joining units 5 in FIG. 14 is similar to that shown in FIG. 13. The description of the linear joining units 5 in FIG. 13 and the description of the linear joining units 5 in FIG. 9 that is applied to FIG. 13 are also applicable to the example shown in FIG. 14, except that the linear joining units 5 are discontinuous between the first elastic member 3221 and the third elastic member 3223.

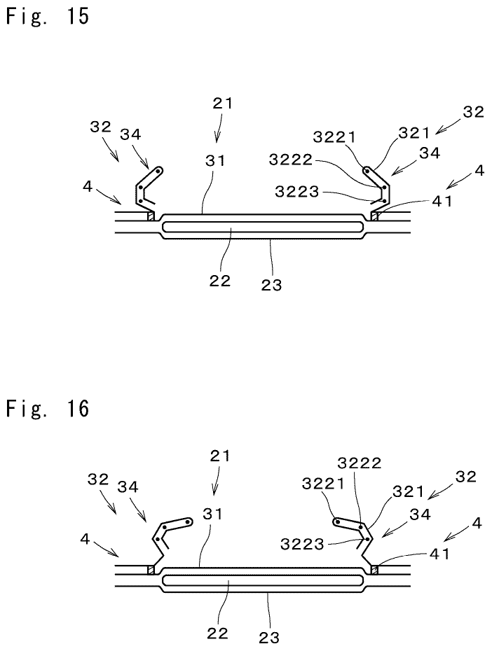

FIG. 15 is a diagram showing a section of the standing gather sheets 32 as another example as in FIG. 3. In FIG. 15, constituent elements that are identical to those in FIG. 3 are given the same reference signs. Each gather sheet 32 in FIG. 15 stands up from the inner side of the central joining region 41 in the width direction toward the outer side in the width direction and then toward the inner side in the width direction via the third elastic member 3223 and the second elastic member 3222. The first elastic member 3221 is arranged in the vicinity of the inner end of the gather sheet 32 in the width direction. In this way, the gathers 34 may stand up first toward the outer side in the width direction and then toward the inner side. This enlarges the inner space of the gathers 34.

FIG. 16 is a diagram showing a section of the standing gather sheets 32 as yet another example as in FIG. 3. In FIG. 16, constituent elements that are identical to those in FIG. 3 are given the same reference signs. Each gather sheet 32 in FIG. 16 strands up from the inner side of the central joining region 41 in the width direction toward the inner side in the width direction and stands up toward the outer side and then toward the inner side in the width direction via the third elastic member 3223 and the second elastic member 3222. The first elastic member 3221 is arranged in the vicinity of the inner end of the gather sheet 32 in the width direction. In this way, the gathers 34 may stand up once toward the inner side in the width direction and then toward the outer side and then toward the inner side. This enlarges the inner space of the gathers 34. Since the gather sheets 32 shown in FIGS. 15 and 16 have the gathers 34 that are bent as viewed in section, it is preferable to adopt the linear joining units 5 that are bent as shown in FIG. 13 or 14.

The structure and application of the gather sheets 32 may be modified in various ways. The stretchable sheet structure of the gather sheets 32 may be used in various places other than the gathers 34 of the absorbent product 1 that come in contact with the groin of a person. In the above description, the stretchable sheet structure is a structure formed by the inside sheet 331, the outside sheet 332, the elastic members, and the linear joining units 5, and is a structure that stretches in a stretching direction with a stretching force exerted thereon and shrinks when the stretching force is released. The gathers 34 may be regarded as the stretchable sheet structure itself, or may be regarded as being included in the stretchable sheet structure. An object that includes the stretchable sheet structure can be expressed as a “stretchable sheet structural object.” The gathers 34 and the gather sheets 32 in the above description also serve as stretchable sheet structural objects.

The absorbent product having the above-described stretchable sheet structure is not limited to an auxiliary absorbent pad, and may be a pants-type disposable diaper, an open-type disposable diaper that is set to around the waist with tapes, an auxiliary absorbent pad arranged in a diaper or a diaper-shaped cover, an urine pad, or any of other various absorbent products that receive body wastes from a wearer. The stretchable sheet structure may be used as, for example, a structure that forms gathers around the waist of a disposable diaper or as a structure that forms gathers around legs of the disposable diaper. In this way, the inside sheet 331 and the outside sheet 332 described above, which sandwich the elastic members, may serve as sheets for use in other applications, and these sheets that sandwich the elastic members in the stretchable sheet structure may be generally referred to as the “first sheet” and the “second sheet.” The number of elastic members is also not limited to three and is at least two, or may be four or more. That is, the stretchable sheet structure includes at least the first sheet, the second sheet overlaid on the first sheet, the first elastic member positioned between the first sheet and the second sheet and extending linearly in the stretching direction, and the second elastic member positioned between the first sheet and the second sheet and extending linearly in the stretching direction while being spaced apart from the first elastic member.

The first sheet and the second sheet do not necessarily have to be obtained by folding a single sheet, and they may be individual sheets. The first sheet and the second sheet may be sheets of the same material, or may be sheets of different materials. The first sheet and the second sheet are not limited to nonwoven fabrics. They may be plastic films or sheets made of any other material, and may have an affinity for water or have hydrophobic properties.

While the first sheet and the second sheet are stretched flat in the stretching direction, the first sheet and the second sheet are joined together by a plurality of linear joining units aligned in the stretching direction. Like the linear joining units 5 shown in, for example, FIGS. 9 and 11 to 14, the shape of the linear joining units may be linear or may be bent. Each linear joining unit may be bent at two or more points, or may be curved. For example, each linear joining unit may have an approximately horizontally-oriented C-shape, or may have an approximately horizontally-oriented S-shape. The linear joining units do not necessarily have to have perfectly the same shape. For example, the shapes of the linear joining units may change gradually in the stretching direction. In this way, although each of the linear joining units exists in the direction intersecting with the stretching direction between the first elastic member and the second elastic member, the direction of intersection does not necessarily have to be constant as long as it does not become parallel to the stretching direction.

Preferably, as shown in FIGS. 9 and 11 to 14, the direction of intersection is inclined with respect to the direction perpendicular to the stretching direction. Accordingly, it is possible to provide a stretchable sheet structure that has appropriate stiffness while forming gathers with orderly ridges and grooves. The direction of interaction, i.e., the direction of extension of the linear joining units, may preferably be inclined at an angle of greater than or equal to 3 degrees and less than or equal to 25 degrees with respect to the width direction and more preferably at an angle of greater than or equal to 5 degrees and less than or equal to 20 degrees. When the direction of intersection changes in the width direction, the direction of extension of the linear joining units may include a portion that is parallel to the width direction. In the case where the linear joining units are bent as in FIGS. 13 and 14, in order to allow the linear joining units to become easily bent on the end side of the gathers, it is preferable that the inclination angle of the direction of extension of each linear joining unit with respect to the width direction between the first elastic member on the end side and the second elastic member is greater than the inclination angle of the direction of extension of the linear joining unit with respect to the width direction between the second elastic member and the third elastic member (that are not on the end side). In this case, the inclination angle of the direction of extension of each linear joining unit with respect to the width direction between the first elastic member and the second elastic member may be on the same side as or may be on the different side from the inclination angle of the direction of extension of the linear joining unit with respect to the width direction between the second elastic member and the third elastic member.

The language “while the first sheet and the second sheet are stretched flat in the stretching direction” refers to a state in which the stretchable sheet structure is stretched until the ridges and grooves of the gathers disappear. In this state, each elastic member is in a stretched state.

As described previously, the number of elastic members included in the stretchable sheet structure is at least two. Thus, if two elastic members are focused on and generally referred to as the “first elastic member” and the “second elastic member,” respectively (of course, these elastic members are not limited to the first elastic member 3221 and the second elastic member 3222 shown in FIG. 3), the first sheet or the second sheet has bulges between the linear joining units to form the gathers while the first elastic member and the second elastic member shrink. The term “bulges” as used herein refer to the case where the sheets are bowed outward in order to form the ridges and grooves, and for example, may refer to the case where part of the first sheet or part of the second sheet is spaced away from the surface including the linear joining units. Note that the first sheet and the second sheet may both have bulges between the linear joining units. At least one of the first sheet and the second sheet between the linear joining units does not necessarily have to have “perfect” bulges between the linear joining units, and may have substantial bulges between the linear joining units.

Focusing on the first elastic member and the second elastic member, it is preferable as described previously that the joining width of each linear joining unit at the first intersecting position of the linear joining unit and the first elastic member and the joining width of the linear joining unit at the second intersecting position between the linear joining unit and the second elastic member are greater than the joining width of the linear joining unit at any position between the first intersecting position and the second intersecting position. As described previously, the term “joining width” may be defined variously as long as the width of the linear joining units is appropriately determined. By increasing the joining width at the intersecting positions, it is possible to ensure the flexibility of the gathers while making strong the joints between the first and second sheets and the first and second elastic members. By increasing the joining width only at the intersecting positions, it is possible to acquire cushioning properties of the gathers without excessively increasing the stiffness of the gathers and thereby to provide gathers that are comfortable to touch. As a result, it is possible to provide a stretchable sheet structure that has appropriate stiffness while forming gathers with orderly ridges and grooves.

Preferably, the joining widths at the first and second intersecting positions are 5 times or more and 12 times or less of the joining width at any position between the first intersecting position and the second intersecting position, and more preferably 7 times or more and 10 times or less of the joining width at any position between the first and second intersecting positions. This facilitates the formation of sufficiently large ridges and grooves.

The techniques used to ensure the flexibility of the gathers, namely the technique for inclining the linear joining units with respect to the direction perpendicular to the stretching direction and the technique for increasing the joining width of the linear joining units at the intersecting positions of the linear joining unis and the elastic members, are techniques independent of each other, and there is no need to always adopt both of the techniques to the stretchable sheet structure.

Each linear joining unit intersects with the first elastic member and the second elastic member at the first intersecting position and the second intersecting position, respectively. As described previously, at the positions intersecting with the elastic members, the first sheet and the second sheet are not joined completely due to the intervention of the elastic members. That is, the “intersection” of the linear joining units and the elastic members means conceptual intersection of the linear joining units and the elastic members and does not mean overlapping of the joining regions of the first and second sheets with the elastic members. As described previously, the linear joining units may exist continuously in the aforementioned intersecting direction, or may exist discontinuously. In the case where the gathers are to be provided with a certain degree of stiffness, it is preferable that the linear joining units are continuous between the elastic members. This, for example, suppresses leakage of body wastes such as urine when the gathers come in contact with the groin. The linear joining units refer to the joining regions that contribute to the formation of the ridges and grooves. In the cases of the examples shown in FIGS. 9 and 11 to 14, the linear joining units primarily refer to the joining regions between the first elastic member 3221 and the third elastic member 3223 among the joining regions between the inside sheet 331 and the outside sheet 332, but they may also include the regions 501 and 502. At least the joining regions between adjacent elastic members are regarded as the linear joining units.

At the linear joining units, the first sheet and the second sheet are preferably joined together by welding. However, an adhesive may be used to join the sheets, or welding and an adhesive may be used in combination to join the sheets. When joined together by welding, the first sheet and the second sheet are preferably water-repellent or liquid-impermeable hydrophobic fabrics formed of hydrophobic fiber. For example, the first sheet and the second sheet may be formed of materials of the same type or different types selected from, for example, a spun-bonded nonwoven fabric, a melt-blown nonwoven fabric, or an SMS nonwoven fabric. The use of welding allows reliable formation of the ridges and grooves and allows stable formation of the beautiful gathers with ridges and grooves aligned uniformly in the stretching direction. Besides, the stable formation of the ridges and grooves reduces the possibility that the elastic members or the linear joining units (i.e., welded regions) may come in contact with the skin, and accordingly improves the touch of the gathers.

In the case where the number of elastic members is three as in the examples shown in FIGS. 9 and 11 to 14 and when these elastic members are generally referred to as the “first elastic member,” the “second elastic member,” and the “third elastic member” (of course, these elastic members are not limited to the first elastic member 3221, the second elastic member 3222, and the third elastic member 3223 shown in FIG. 3), the above description focusing on the first elastic member and the second elastic member described above is applicable to the description about the second elastic member and the third elastic member. That is, each of the linear joining units exists in a direction intersecting with the stretching direction between the second elastic member and the third elastic member, and each linear joining unit intersects with the second elastic member and the third elastic member. Then, while the first elastic member, the second elastic member, and the third elastic member shrink, ridges and grooves are formed between adjacent ones of the linear joining units to form the gathers.

Preferably, the joining width of each linear joining unit at the second intersecting position of the linear joining unit and the second elastic member and the joining width of the linear joining unit at the third intersecting position of the linear joining unit and the third elastic member are greater than the joining width of the linear joining unit at any position between the second intersecting position and the third intersecting position. The other descriptions focusing on the first elastic member and the second elastic member described above are also applicable to the descriptions about the second elastic member and the third elastic member.

The stretchable sheet structure described above may be used in products other than absorbent products. The stretchable sheet structure can be employed in various products that are desired to have gathers orderly formed.

The configurations of the above-described preferred embodiment and variations may be appropriately combined as long as there are no mutual inconsistencies.

While the invention has been shown and described in detail, the foregoing description is in all aspects illustrative and not restrictive. It is therefore understood that numerous modifications and variations can be devised without departing from the scope of the invention.

### REFERENCE SIGNS LIST

1 absorbent product
5 linear joining unit
34 gathers
331 inside sheet (first sheet)
332 outside sheet (second sheet)
333 edge (folding line)
511 first intersecting position
512 second intersecting position
3221 first elastic member
3222 second elastic member
3223 third elastic member

1. A stretchable sheet structure that stretches in a stretching direction with a stretching force exerted thereon and shrinks when said stretching force is released, comprising:
a first sheet;
a second sheet overlaid on said first sheet;
a first elastic member positioned between said first sheet and said second sheet and extending linearly in said stretching direction; and
a second elastic member positioned between said first sheet and said second sheet and extending linearly in said stretching direction while being spaced apart from said first elastic member,
wherein, while said first sheet and said second sheet are stretched flat in said stretching direction, said first sheet and said second sheet are joined together by a plurality of linear joining units aligned in said stretching direction,
each linear joining unit intersects with said first elastic member and said second elastic member,
a joining width of said each linear joining unit at a first intersecting position of said each linear joining unit and said first elastic member and a joining width of said each linear joining unit at a second intersecting position of said each linear joining unit and said second elastic member are greater than a joining width of said each linear joining unit at any position between said first intersecting position and said second intersecting position, and
while said first elastic member and said second elastic member shrink, said first sheet or said second sheet has bulges between said plurality of linear joining units to form gathers.

2. A stretchable sheet structure that stretches in a stretching direction with a stretching force exerted thereon and shrinks when said stretching force is released, comprising:
a first sheet;
a second sheet overlaid on said first sheet;
a first elastic member positioned between said first sheet and said second sheet and extending linearly in said stretching direction; and
a second elastic member positioned between said first sheet and said second sheet and extending linearly in said stretching direction while being spaced apart from said first elastic member,
wherein, while said first sheet and said second sheet are stretched flat in said stretching direction, said first sheet and said second sheet are joined together by a plurality of linear joining units aligned in said stretching direction,
each of said plurality of linear joining units exists in an intersecting direction intersecting with said stretching direction, between said first elastic member and said second elastic member, the intersecting direction being inclined with respect to a direction perpendicular to said stretching direction,
each linear joining unit intersects with said first elastic member and said second elastic member, and
while said first elastic member and said second elastic member shrink, said first sheet or said second sheet has bulges between said plurality of linear joining units to form gathers.

3. The stretchable sheet structure according to claim 2, wherein
a joining width of said each linear joining unit at a first intersecting position of said each linear joining unit and said first elastic member and a joining width of said each linear joining unit at a second intersecting position of said each linear joining unit and said second elastic member are greater than a joining width of said each linear joining unit at any position between said first intersecting position and said second intersecting position.

4. The stretchable sheet structure according to claim 1, wherein
each of said plurality of linear joining units continuously exists in an intersecting direction intersecting with said stretching direction, between said first elastic member and said second elastic member.

5. The stretchable sheet structure according to claim 1, wherein
said first sheet and said second sheet are joined together at said each linear joining unit by welding.

6. The stretchable sheet structure according to claim 1, wherein
said first sheet and said second sheet are a single sheet folded along a folding line, while said first sheet and said second sheet are stretched flat in said stretching direction, said first elastic member is positioned in close proximity to and in parallel with said folding line, and
said each linear joining unit does not overlap with said folding line.

7. The stretchable sheet structure according to claim 1, further comprising:
a third elastic member positioned between said first sheet and said second sheet and extending linearly in said stretching direction while being spaced apart from said second elastic member on a side of said second elastic member opposite to said first elastic member,
wherein each of said plurality of linear joining units exits in an intersecting direction intersecting with said stretching direction, between said second elastic member and said third elastic member,
said each linear joining unit intersects with said third elastic member,
said gathers are formed while said first elastic member, said second elastic member, and said third elastic member shrink, and
said each linear joining unit is linear while said first sheet and said second sheet are stretched flat in said stretching direction.

8. The stretchable sheet structure according to claim 1, further comprising:
a third elastic member positioned between said first sheet and said second sheet and extending linearly in said stretching direction while being spaced apart from said second elastic member on a side of said second elastic member opposite to said first elastic member,
wherein each of said plurality of linear joining units exists in an intersecting direction intersecting with said stretching direction, between said second elastic member and said third elastic member,
said each linear joining unit intersects with said third elastic member,
said gathers are formed while said first elastic member, said second elastic member, and said third elastic member shrink, and
while said first sheet and said second sheet are stretched flat in said stretching direction, said each linear joining unit is bent at a position of said second elastic member.

9. The stretchable sheet structure according to claim 8, wherein
an angular direction in which said each linear joining unit is inclined with respect to the direction perpendicular to said stretching direction between said first elastic member and said second elastic member is opposite to an angular direction in which said each linear joining unit is inclined with respect to the direction perpendicular to said stretching direction between said second elastic member and said third elastic member.

10. The stretchable sheet structure according to claim 7, wherein
while said first sheet and said second sheet are stretched flat in said stretching direction, said first sheet and said second sheet have an edge that exists along and in close proximity to said first elastic member on a side of said first elastic member opposite to said second elastic member, and
an interval between said first elastic member and said second elastic member is greater than an interval between said second elastic member and said third elastic member.

11. The stretchable sheet structure according to claim 8, wherein
while said first sheet and said second sheet are stretched flat in said stretching direction, said first sheet and said second sheet have an edge that exists along and in close proximity to said first elastic member on a side of said first elastic member opposite to said second elastic member, and
an interval between said first elastic member and said second elastic member is greater than an interval between said second elastic member and said third elastic member.

12. The stretchable sheet structure according to claim 1, wherein
while said first sheet and said second sheet are stretched flat in said stretch direction, a range of existence of said each linear joining unit in said stretching direction overlaps in part with a range of existence of another linear joining unit in said stretching direction, the another linear joining unit being adjacent to said each linear joining unit.

13. A disposable absorbent product that receives a body waste from a wearer, comprising:
the stretchable sheet structure according to claim 1.

14. The disposable absorbent product according to claim 13, wherein said gathers come in contact with a groin of said wearer.

15. The stretchable sheet structure according to claim 2, wherein
each of said plurality of linear joining units continuously exists in an intersecting direction intersecting with said stretching direction, between said first elastic member and said second elastic member.

16. The stretchable sheet structure according to claim 2, wherein
said first sheet and said second sheet are joined together at said each linear joining unit by welding.

17. The stretchable sheet structure according to claim 2, wherein
said first sheet and said second sheet are a single sheet folded along a folding line,
while said first sheet and said second sheet are stretched flat in said stretching direction, said first elastic member is positioned in close proximity to and in parallel with said folding line, and
said each linear joining unit does not overlap with said folding line.

18. The stretchable sheet structure according to claim 2, further comprising:
a third elastic member positioned between said first sheet and said second sheet and extending linearly in said stretching direction while being spaced apart from said second elastic member on a side of said second elastic member opposite to said first elastic member,
wherein each of said plurality of linear joining units exits in an intersecting direction intersecting with said stretching direction, between said second elastic member and said third elastic member,
said each linear joining unit intersects with said third elastic member,
said gathers are formed while said first elastic member, said second elastic member, and said third elastic member shrink, and
said each linear joining unit is linear while said first sheet and said second sheet are stretched flat in said stretching direction.

19. The stretchable sheet structure according to claim 2, further comprising:
a third elastic member positioned between said first sheet and said second sheet and extending linearly in said stretching direction while being spaced apart from said second elastic member on a side of said second elastic member opposite to said first elastic member,
wherein each of said plurality of linear joining units exists in an intersecting direction intersecting with said stretching direction, between said second elastic member and said third elastic member,
said each linear joining unit intersects with said third elastic member,
said gathers are formed while said first elastic member, said second elastic member, and said third elastic member shrink, and
while said first sheet and said second sheet are stretched flat in said stretching direction, said each linear joining unit is bent at a position of said second elastic member.

20. The stretchable sheet structure according to claim 19, wherein
an angular direction in which said each linear joining unit is inclined with respect to the direction perpendicular to said stretching direction between said first elastic member and said second elastic member is opposite to an angular direction in which said each linear joining unit is inclined with respect to the direction perpendicular to said stretching direction between said second elastic member and said third elastic member.

21. The stretchable sheet structure according to claim 18, wherein
while said first sheet and said second sheet are stretched flat in said stretching direction, said first sheet and said second sheet have an edge that exists along and in close proximity to said first elastic member on a side of said first elastic member opposite to said second elastic member, and
an interval between said first elastic member and said second elastic member is greater than an interval between said second elastic member and said third elastic member.

22. The stretchable sheet structure according to claim 19, wherein
while said first sheet and said second sheet are stretched flat in said stretching direction, said first sheet and said second sheet have an edge that exists along and in close proximity to said first elastic member on a side of said first elastic member opposite to said second elastic member, and
an interval between said first elastic member and said second elastic member is greater than an interval between said second elastic member and said third elastic member.

23. The stretchable sheet structure according to claim 2, wherein
while said first sheet and said second sheet are stretched flat in said stretch direction, a range of existence of said each linear joining unit in said stretching direction overlaps in part with a range of existence of another linear joining unit in said stretching direction, the another linear joining unit being adjacent to said each linear joining unit.

24. A disposable absorbent product that receives a body waste from a wearer, comprising:
the stretchable sheet structure according to claim 2.

25. The disposable absorbent product according to claim 24, wherein
said gathers come in contact with a groin of said wearer.
